(19) United States
(12) Patent Application Publication
(10) Pub. No.: US 2024/0050685 A1
(43) Pub. Date: Feb. 15, 2024

(54) AIRFLOW SPLITTER, AIRFLOW SPLITTING ASSEMBLY, AND HOMOGENEOUS FLOW PRODUCING DEVICE

(71) Applicant: RESVENT MEDICAL TECHNOLOGY CO., LTD, Shenzhen (CN)

(72) Inventors: Kai Qu, Shenzhen (CN); Rongrong Zhu, Shenzhen (CN)

(73) Assignee: RESVENT MEDICAL TECHNOLOGY CO., LTD, Shenzhen (CN)

(21) Appl. No.: 18/365,957

(22) Filed: Aug. 4, 2023

(30) Foreign Application Priority Data
Aug. 9, 2022 (CN) 202210949655.8

(51) Int. Cl. A61M 16/08

(52) U.S. Cl. CPC A61M 16/0875; A61M 16/0833

(57) ABSTRACT

An airflow splitter is provided. The airflow splitter includes an inlet, an outlet, at least two splitting member. The splitting member includes a small aperture end and a large aperture end opposite to each other, the small aperture ends of all the splitting members face the inlet, and the large aperture ends of all the splitting members face the outlet. A flow pathway is defined between the small aperture ends and the large aperture ends. The at least two splitting members are sleeved sequentially. In two adjacent splitting members, the large aperture end of one splitting member is located in the small aperture end of the other splitting member. An annular gap is formed between the two adjacent splitting members, and the annular gap is further communicated with the flow pathway.

### CROSS REFERENCE TO RELATED APPLICATION

This non-provisional patent application claims priority under 35 U.S.C. § 119 from Chinese Patent Application No. 2022109496558 filed on Aug. 9, 2022, the entire content of which is incorporated herein by reference.

### TECHNICAL FIELD

The disclosure relates to medical instruments, and in particular relate to an airflow splitter, an airflow splitting assembly, and a homogeneous flow producing device.

### BACKGROUND

Ventilator or anesthesia machines define a gas pathway referred to as a gas delivery tube which includes a small diameter tube with a smaller diameter, and a large diameter tube with a large-diameter communicating with the small diameter tube via an expanded tube. When air flows from the small diameter tube to the large diameter tube, non-homogeneous flow may occur, and forward flow at the center of the pathways and back flow at the edge of the pathways may also occur, which leads to inaccurate flow measurement of a flow sensor in the air pathway.

Typically, in order to realize homogeneous flow, a strainer is placed at the expanded tube of the pathway, and the homogeneous flow is better based on the pressure difference between the air in front and rare of the strainer. However, one strainer only bring an un-obvious effect of the homogeneous flow, and there need more filters to ensure the accuracy of the flow sensor measurement. but when the air flows through the strainers, the strainers will cause a large pressure drop, and a maximum flow rate of the airflow will be affected.

Therefore, there is room for promotion in airflow splitter technology.

### SUMMARY

In a first aspect, an airflow splitter is provided. The airflow splitter includes an inlet, an outlet, at least two splitting member. The splitting member includes a small aperture end and a large aperture end opposite to each other, the small aperture ends of all the splitting members face the inlet, and the large aperture ends of all the splitting members face the outlet. A flow pathway is defined between the small aperture ends and the large aperture ends. The at least two splitting members are sleeved sequentially. In two adjacent splitting members, the large aperture end of one splitting member is located in the small aperture end of the other splitting member. An annular gap is formed between the two adjacent splitting members, and the annular gap is further communicated with the flow pathway.

In a second aspect, an airflow splitting assembly is provided. The airflow splitting assembly includes a strainer and an airflow splitter. The airflow splitter includes an inlet, an outlet, at least two splitting member. The splitting member includes a small aperture end and a large aperture end opposite to each other, the small aperture ends of all the splitting members face the inlet, and the large aperture ends of all the splitting members face the outlet. A flow pathway is defined between the small aperture ends and the large aperture ends. The at least two splitting members are sleeved sequentially. In two adjacent splitting members, the large aperture end of one splitting member is located in the small aperture end of the other splitting member. An annular gap is formed between the two adjacent splitting members, and the annular gap is further communicated with the flow pathway.

In a third aspect, a homogeneous flow producing device is provided. The homogeneous flow producing device includes a main body, a first tube, a second tube, an expansion tube, and an airflow splitter. The main body defines a gas output mouth. The first tube is attached to the main body and communicated with the gas output mouth. The second tube has a diameter lager than that of the first tube. The expansion tube has a first end, a second end opposite to each other, and defines an accommodating cavity between the first end and the second end. The first end is connected to the first tube, and the second end is connected to the second tube. The airflow splitter is received in the accommodating cavity. The airflow splitter includes an inlet facing to the first end, an outlet facing to the second end, and at least two splitting member. The splitting member includes a small aperture end and a large aperture end opposite to each other, the small aperture ends of all the splitting members face the inlet, and the large aperture ends of all the splitting members face the outlet. A flow pathway is defined between the small aperture ends and the large aperture ends. The at least two splitting members are sleeved sequentially. In two adjacent splitting members, the large aperture end of one splitting member is located in the small aperture end of the other splitting member. An annular gap is formed between the two adjacent splitting members, and the annular gap is further communicated with the flow pathway.

As described above, the airflow splitter includes at least two splitting members sleeved one after one, and each two adjacent splitting members forms an annular gap there between that enable a part of the gas flow into each gap between each two adjacent splitting members in order, and then flow together in the flow pathway of the corresponding splitting member, which may the gas be splitted and come together many time before flow out of the airflow splitter, as a result, it ensures that the gas flow out of the airflow splitter flows homogeneously.

In order to more clearly explain the technical scheme in the embodiments of the present application or the prior art, the following is a brief introduction of the drawings required to be used in the description of the embodiments or the prior art. Obviously, the drawings described below are only some embodiments of the present application. Other drawings can also be obtained based on the structures shown in these drawings.

### BRIEF DESCRIPTION OF THE DRAWINGS

FIG. 1 illustrates a perspective view of an airflow splitter in accordance with an embodiment.

FIG. 2 illustrates a cross-sectional view of the airflow splitter in accordance with an embodiment

FIG. 3 illustrates an exploded schematic diagram of a airflow splitting assembly in accordance with an embodiment.

FIG. 4 illustrates a schematic cross-sectional view of a homogeneous flow producing assembly in accordance with an embodiment.

FIG. 5 illustrates a schematic diagram of the gas flow state in the homogeneous flow producing assembly shown in FIG. 4.

FIG. 6 illustrates a schematic cross-sectional view of a homogeneous flow producing device in accordance with an embodiment.

FIG. 7 illustrates an exploded schematic view of the airflow splitter and the strainer assembly in the homogeneous flow producing device accordance with an embodiment.

FIG. 8 illustrates a schematic diagram of a gas flow state in a typical gas pathway.

The realization, functional features and advantages of the present application will be further described in conjunction with the embodiments and with reference to the accompanying drawings.

### DETAILED DESCRIPTION OF THE EMBODIMENTS

The technical scheme of the disclosure will be clearly and completely described below in combination with the accompanying drawings. Obviously, the described embodiments are part of the embodiments of the disclosure, not all of them. Based on the embodiments of the disclosure, all other embodiments obtained by those skilled in the art without creative work belong to the protection scope of the disclosure.

In the description of the disclosure, it should be noted that the terms used herein are only for the purpose of describing specific embodiments and are not intended to limit the disclosure. The singular forms “one”, “one” and “this” as used herein also include the plural unless expressly stated by the context. When the terms “include” and/or “include” are used, it is intended to indicate the existence of the feature, integer, step, operation, element and/or component, and does not exclude the existence or addition of one or more other features, integer, step, operation, element, component, and/or other combinations. The term “and/or” includes any and all combinations of one or more related listed items. The azimuth or positional relationship indicated by the terms “center”, “up”, “down”, “left”, “right”, “vertical”, “horizontal”, “inside”, “outside” is based on the azimuth or positional relationship shown in the attached drawings only for the convenience of describing the disclosure and simplifying the description, rather than indicating or implying that the device or element referred to must have a specific azimuth It is constructed and operated in a specific orientation and therefore cannot be understood as a limitation of the present disclosure. The terms “first” and “second” are used only for descriptive purposes and cannot be understood as indicating or implying relative importance. The terms “installation”, “connection” and “connection” shall be understood in a broad sense. For example, it can be fixed connection, removable connection or integrated connection; It can be directly connected, indirectly connected through an intermediate medium, or the connection between the two elements. For those skilled in the art, the specific meaning of the above terms in the disclosure can be understood in specific circumstances.

In addition, some diagrams in this specification are flow charts for illustrating methods. It should be understood that each block in these flowcharts and the combination of blocks in these flowcharts can be implemented by computer program instructions. These computer program instructions may be loaded onto a computer or other programmable device to form a machine so that the instructions executed on the computer or other programmable device form a structure for implementing the functions specified in the flowchart block. These computer program instructions may also be stored in a computer-readable memory, which may instruct a computer or other programmable device to work in a specific manner so that the instructions stored in the computer-readable memory form an article containing an instruction structure for implementing the functions specified in the flowchart block. The computer program instructions can also be loaded on a computer or other programmable device to perform a series of operation steps on the computer or other programmable device to form a process implemented by the computer, so that the instructions executed on the computer or other programmable device provide steps for implementing the functions specified in the flowchart block.

Accordingly, the blocks in each flowchart support a combination of structures for performing the specified function and a combination of steps for performing the specified function. It should also be understood that each block in the flowchart and the combination of blocks in the flowchart can be implemented by a special hardware based computer system performing the specified functions or steps, or a combination of special hardware and computer instructions.

In addition, the technical features involved in different embodiments of the disclosure described below can be combined with each other as long as they do not constitute a conflict with each other.

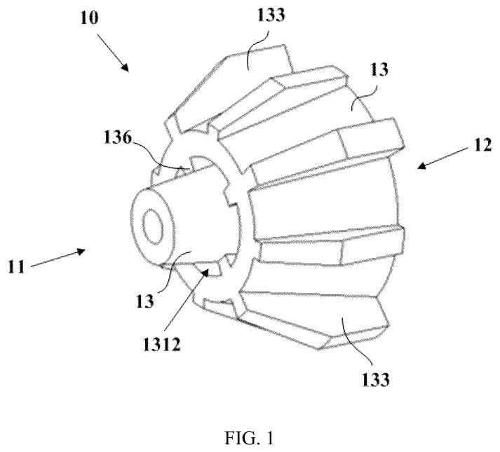

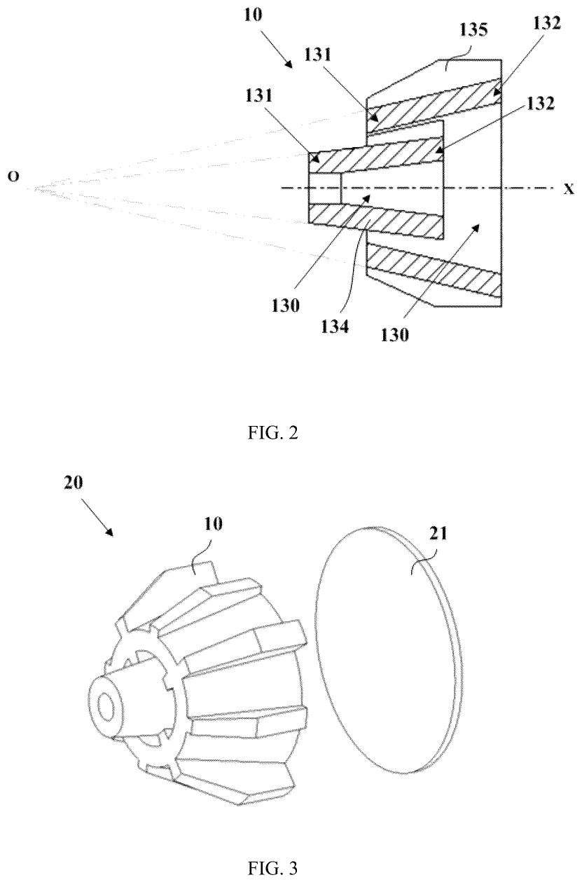

Referring to FIG. 1 and FIG. 2, FIG. 1 illustrates a perspective view of an airflow splitter 10. FIG. 2 illustrates a cross-sectional view of the airflow splitter 10. The airflow splitter 10 is configured to divert the gas flowing from a small diameter tube to a large diameter tube, which has a diameter lager than that of the small diameter tube. The airflow splitter 10 includes an inlet 11 and an outlet 12 opposite to the inlet 11, the inlet 11 faces the small tub and allows the gas to flow into the small tub, and the outlet 12 faces the tub and allows the gas to flow out of the large tub. In other words, the gas flow form the inlet to the outlet 12. In this embodiment, the airflow splitter 10 is substantially in a cone shape.

The airflow splitter 10 further includes at least two splitting member 13, the at least two splitting members 13 are sleeved together one after one. It is understood that the at least two splitting members 13 are sleeved sequentially one after one from the inside to the outside.

In this embodiment, the splitting member 13 is substantially in a truncated-cone-shape. Each splitting member 13 includes a small aperture end 131 and a large aperture end 132 opposite each other. It is understood that for one splitting member 13, a diameter of the small aperture end 131 of is less than a diameter of the large aperture end 132. The small aperture ends 131 of all the splitting members 13 are oriented towards the inlet 11, and the large aperture ends 132 of all the splitting members 13 are oriented towards the outlet 12.

A flow pathway 130 is defined between the small aperture end 131 and the large aperture end 132, and the flow pathway 130 is in truncated-cone-shape. It is understood that the diameter of the flow pathway 130 located at the small aperture end 131 is smaller than the diameter of the flow pathway 130 located at the large aperture end 132. In this case, a central axis X of the flow pathway of all splitting members 13 are coincide, and the extension lines of the outer walls of all splitting members 13 intersect at the same point O. That is, all splitting members 13 are coaxial and confocal.

Taking two adjacent splitting members 13 as an example, the large aperture end 132 of one splitting member 13 is inserted within the small aperture end 131 of the other splitting member 13. That is, the large aperture end 132 of one splitting member 13 is accommodated in the flow pathway 130 of the other splitting member 13. It will be understood that the large aperture end 132 of the inner splitting member 13 is received within the small aperture end 131 of the outer splitting member 13, the inner splitting member 13 and the outer splitting member 13 are arranged adjacent to each other. In this embodiment, an annular gap 1312 is formed between the inner wall of the small aperture end 131 of the other splitting member 13 and the outer wall of the large aperture end 1325 of the one splitting member 13, and the annular gap 1312 is communicated with the flow pathway 130. That is, in two adjacent splitting members 13, an annular gap 1312 is formed between the outer wall of the large aperture end 132 of the inner splitting member 13 and the inner wall of the small aperture end 131 of the outer splitting member 13. It is understood that if a quantity of the splitting members 13 is n, the quantity of the annular gaps 1312 is n−1. In each two adjacent splitting members, the splitting member 13 located at inside will be referred to as an inner splitting member or a first member, the splitting member 13 located at outside will be referred to as an inner splitting member or a second member.

In this embodiment, because all splitting members 13 are sequentially stackably sleeved together, the flow pathway 130 of all splitting member 13 are communicated and form a main flow pathway in the center of the airflow splitter 10. The annular gap 1312 formed between two adjacent splitting member 13 is communicated to the flow pathway 130 of the outer splitting member 13 of the two adjacent splitting member 13 and forms a plurality of auxiliary flow pathways. Each of the auxiliary flow pathways is communicated to the main flow pathway. If the quantity of the splitting member 13 is n, the quantity of the auxiliary flow pathways is n−1.

Further, an inclination of an outer surface of the inner splitting member 13 is less than that of the outer splitting member 13. It will be understood that for the splitting members 13 in a stackably sleeved manner, central angles corresponding to extension lines of each outer surface gradually increases. That is to say, one splitting member 13 is located in a more inward position of the airflow splitter 10, the central angle of the one splitting member 13 becomes smaller, and the one splitting member 13 is located in a more outward position of the airflow splitter 10, the central angle of the one splitting member 13 becomes larger.

Further, the small aperture end 131 of the inner splitting member 13 has a smaller diameter than that of the outer splitting member 13. The diameter of the large aperture end 132 of the inner splitting member 13 is smaller than of the large aperture end 132 of the outer splitting member 13. In this embodiment, limiting ribs 136 are located in an inner wall of the outer splitting member 13 of each two adjacent splitting members 13, the inner wall faces to the inner splitting member 13 of each of the two adjacent splitting members correspondingly. All the limiting ribs 136 are placed along a central axis X of the flow pathway. The limiting ribs 136 protrude from the inner wall towards to the faced splitting member 13. The limiting ribs 136 located in the inner wall of the outer splitting member 13 abuts against an outer wall of the inner splitting member 13 that the inner splitting member 13 is fixed via the outer splitting member 13. Furthermore, the inner splitting member 13 is placed into the outer splitting member 13 via the large aperture end 132 of the outer splitting member 13, and moved towards the small aperture end 131 of outer splitting member 13 until the inner splitting member 13 abuts against the limiting ribs 136 of the outer splitting member 13, as a result, the inner splitting member 13 is secured to the outer splitting member 13.

Further, the small aperture end 131 of the inner splitting member 13 extends in a direction away from the outer splitting member 13, and protrudes out of the outer splitting member 13. Correspondingly, the large aperture end 132 of the outer splitting member 13 extends in a direction away from the inner splitting member 13 and protrudes out of the inner splitting member 13. In other words, when the splitting members 13 are stackably sleeved, the small aperture end 131 and the large aperture end 132 of the each splitting member 13 are gradually placed backward along the gas flow direction. In this embodiment, all splitting member 13 have the same height along the central axis X of the flow pathway. Accordingly, the splitting member 13 located in more inward position becomes closer to the inlet 11 of the airflow splitter 10, and the splitting member 13 located in more outward position becomes closer to the outlet 12 of the airflow splitter 10. It is understood that each two adjacent splitting member 13 are partially nested.

The at least two splitting member 13 include an inner splitting member 13 and an outer splitting member 13 sleeved outside of the inner splitting member 13; or includes an inner splitting member 13, an outer splitting member 13, and one or more middle splitting member 13 located between the inner splitting member 13 and the outer splitting member 13. All the splitting members 13 except the inner splitting member 13, have the limiting ribs 136. That is, the inner splitting member 13 does not has the limiting ribs 136.

In this embodiment, the outer wall of the outer splitting member 13 protrudes a plurality of protrusion ribs 133 at equal intervals. The protrusion ribs 133 protrude from the outer wall of the outer splitting member 13 in a direction away from the outer splitting member 13. The protrusion ribs 133 are parallel to the central axis X of the flow pathway. Some protrusion ribs 133 are opposite to the limiting ribs 136 correspondingly.

The airflow splitter 10 shown in FIGS. 1 and 2 is taken as an example, the airflow splitter 10 includes two splitting member 13 with an inner splitting member 134 and an outer splitting member 135. The outer splitting member 135 includes four limiting ribs 136 and eight protrusion ribs 133, the four protrusion ribs 133 are opposite the four limiting ribs 136 respectively. An annular gap 1312 and an auxiliary flow pathway are formed between the inner splitting member 134 and the outer splitting member 135.

Referring to FIG. 3, an exploded schematic view of an airflow splitting assembly in an embodiment is illustrated. The airflow splitting assembly 20 is located in the flow pathway of a ventilator or anesthesia machine. In this embodiment, the airflow splitting assembly 20 includes the airflow splitter 10 and a strainer 21. In this embodiment, the strainer 21 is placed at the outlet 12 of the airflow splitter 10. It is understood that the airflow splitter 10 is in a cone-like shape, the strainer 21 has a matching circular cross section. The strainer 21 can be fixed to the outer splitting member 13 of the airflow splitter 10 by ultrasonic welding or glue bonding.

In this embodiment, the strainer 21 can be reduce the backflow when the gas flows out of the outlet 12 of the airflow splitter 10, better homogeneous flow, and ensure that the gas passing through the strainer 21 can be flow evenly in the large diameter tube.

The detail structure of the manifold assembly 10 can be referred to the above-mentioned embodiments. The airflow splitting assembly 20 adopts all the technical solutions of all the above embodiments, it has at least all the beneficial effects brought by the technical solutions of the above embodiments, which will not be repeated here.

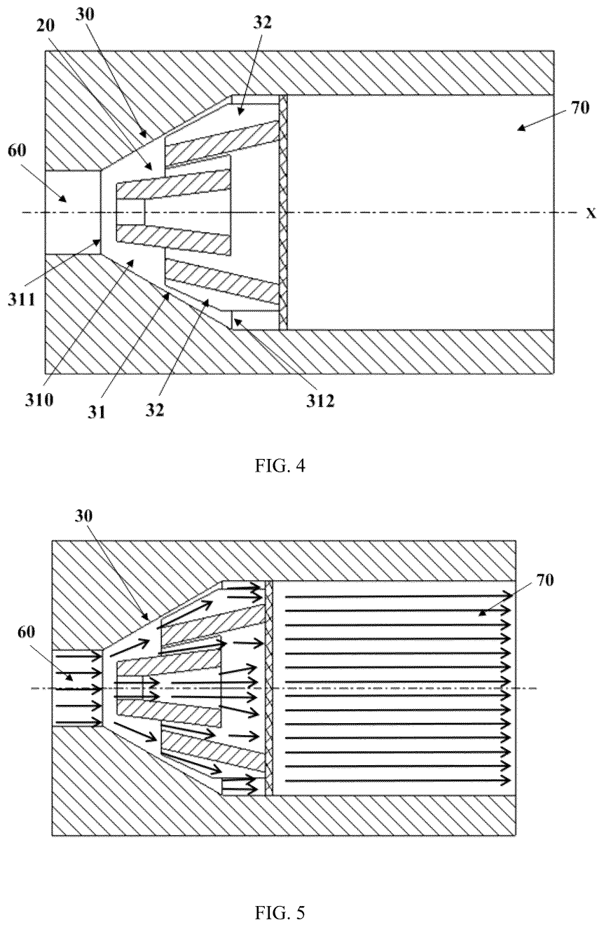

Referring to FIGS. 4 and 5, FIG. 4 illustrates a cross-sectional view of a homogeneous flow producing assembly in accordance with an embodiment, and FIG. 5 illustrates a scheme diagram of a gas flow state in the homogeneous flow producing assembly in accordance with an embodiment. The homogeneous flow producing assembly 30 includes an expansion tube 31 and an airflow splitting assembly 20, the homogeneous flow producing assembly 30 is placed between the small diameter tube 60 and the large diameter tube 70 to enable the small diameter tube 60 communicate the large diameter tube 70.

The expansion tube 31 is in a truncated-cone-shape. The expansion tube 31 includes a first end 311 and a second end 312 opposite to each other, and a diameter of the first end 311 is smaller than that of the second end 312. The first end 311 is communicated to an end of the small diameter tube 60 facing the large diameter tube 70, and the second end 312 is communicated to the end of the large diameter tube 70 facing the small diameter tube 60. An accommodating cavity 310 is defined between the first end 311 and the second end 312.

The airflow splitting assembly 20 is received in the accommodating cavity 310, and the protrusion ribs 133 of the outermost airflow splitter 10 in the airflow splitting assembly 20 engages with a cavity wall around the accommodating cavity 310, so that the airflow splitting assembly 20 is fixed to the expansion tube 31. It can be understood that a size of the airflow splitter 10 is adapted to that of the expansion tube 31. At the same time, since the protrusion ribs 133 protrude from the outer wall of the outer splitting member 13, several airflow passages 32 are formed between the outer wall of the outer splitting member 13 and the cavity wall of the accommodating cavity 310.

In this embodiment, the inlet 11 of the airflow splitter 10 in the airflow splitting assembly 20 faces the first end 311, and the outlet 12 of the airflow splitter 10 faces the second end 312.

When the gas flows from the small diameter tube 60 into the expansion tube 31 through the first end 311, a part of the gas enters the main flow pathway and continues to flow, and the other part of the gas flows along the outer surfaces of all the splitting members 13, and respectively enter into the several auxiliary flow pathways from different annular gaps 1312. That is to say, the other part of the gas flows along the outer surface of one splitting member 13 until the other part of the gas encounters the next one splitting members 13. When the gas encounters the next splitting member 13, a part of the gas flows along the annular gap 1312 between two adjacent splitting members 13, and the other part of the gas flows along the outer surface of next one splitting member 13 until it meets another next splitting member 13. Since, the airflow splitter 10 including a plurality of splitting members 13, the gas continuously flow along the outer surfaces of different splitting members 13 one by one in a manner as described above. All the gas that enters into the airflow splitter 10 from the inlet 11 of the airflow splitter 10 and finally flows out from the outlet 12 of the airflow splitter 10, and then passes through the strainer 21, and finally flows to the second end 312 of the expansion tube 31 and the large diameter tube 70 in order. The homogeneous flow producing assembly 30 can produce homogeneous flow of the gas flowing out of the small diameter tube 60 into the large-diameter tube 70.

The gas flows out along the outer surface of the splitting member will form a negative pressure backflow area on a side of the splitting member 13 away from the inlet 11 due to the incline outer surface of the splitting member 13. The gas flows out along the outer surface of the splitting member meets the gas flows out of the flow pathway 130 of the splitting member 13, and the backflow will be counteracted. Furthermore, the gas flowing out along the flow pathway 130 of one splitting member 13 and the gas flowing out along the outer surface of the one splitting member 13 flow together and the then continue to flow forward along the flow pathway 130 of next splitting member 13 until that encounters a gas flow from the outer surface of the next splitting member 13. That is, all the gas passing through the auxiliary flow pathways and the gas flowing in the main flow pathway may flow together. In addition, after the gas flowing out of the small diameter tube 60 flows into the homogeneous flow producing assembly 30, the gas will be diverted and flow together many times that it ensures the gas flowing in the large-diameter tube 7 to flow in a homogeneous flow manner.

Also taking the airflow splitter having the two splitting members 13 as an example, when the gas flows into the homogeneous flow producing assembly 30 from the small-diameter tube 60, part of the gas flows along the flow pathway 130 of the inner splitting member 134, and the other part of the gas flows along the outer surface of the inner splitting member 134. When encountering the outer splitting member 135, another part of the gas flows into the flow pathway 130 of the outer splitting member 135 from the annular gap 1312 between the inner splitting member 134 and the outer splitting member 135, and the other part of the gas flows along the outer surface of the outer splitting member 135 or flow in the airflow passages 32. The gas flowing into the flow pathway 130 of the outer splitting member 135 and the gas flowing along the flow pathway 130 of the inner splitting member 134 come together at the side of the inner splitting member 134 away from the small aperture end 131. The confluence gas and the gas flowing along the gas flow channel 32 pass through the strainer 21 and then flow into the large-diameter tube 70.

The homogeneous flow producing assembly 30 can ensure that when the gas flows through the expansion diameter tube 31, the gas flows homogeneously along the central axis X of the flow pathway, and the measurement accuracy and stability of the flow sensor in the large diameter tube 70 is significantly improved in the condition that the gas is sufficient.

The detail structure of the airflow splitting assembly 20 can refer to the above-mentioned embodiments. Since the homogeneous flow producing assembly 30 adopts all the technical solutions of all the above-mentioned embodiments, it at least has all the beneficial effects brought by the technical solutions of the above-mentioned embodiments, which will not be repeated.

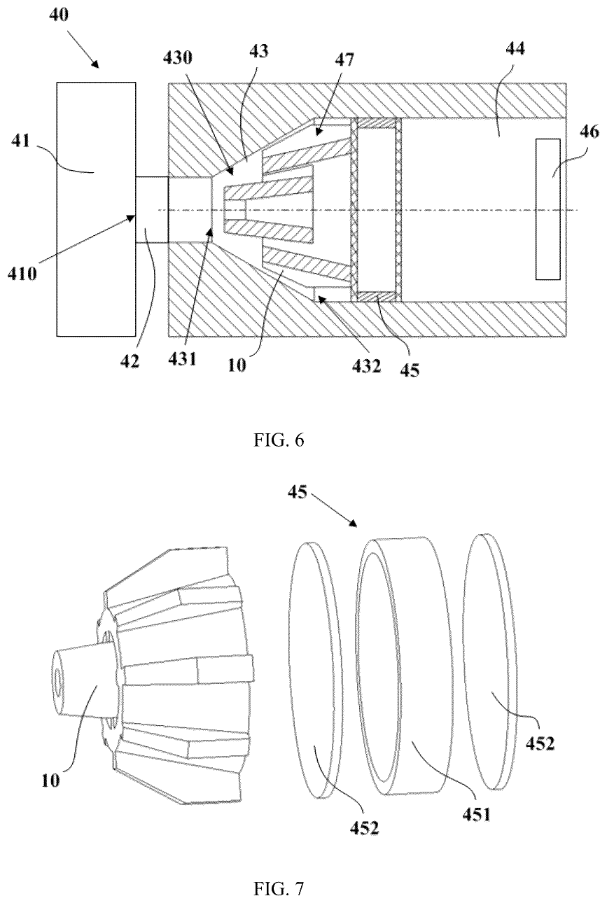

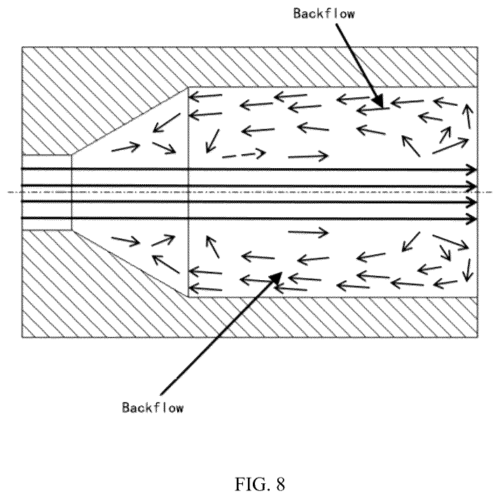

Referring to FIG. 6 and FIG. 7, FIG. 6 is a schematic cross-sectional view of a homogeneous flow producing device in accordance with an embodiment, and FIG. 7 is an exploded schematic view of the airflow splitter and the strainer assembly in the homogeneous flow producing device shown in FIG. 6. the homogeneous flow producing device 40 includes a main body 41, a first tube 42, an expansion tube 43, a second tube 44 and an airflow splitter 10. The main body 41 includes but not limited to a ventilator, an anesthesia machine and so on. In this embodiment, the main body 41 includes a gas output mouth 410. It can be understood that both the ventilator and the anesthesia machine can generate gas, and the generated gas flows out through the gas output mouth 410.

One end of the second tube 44 is connected to the second end 432. The diameter of the second tube 44 is larger than that of the first tube 42.

The airflow splitter 10 is received in the accommodating cavity 430. The protrusion rib 133 of the outer splitting member of the airflow splitter 10 is engaged with the cavity wall of the accommodating cavity 430, so that the airflow splitter 10 is fixed in the expansion tube 43. It can be understood that the size of the airflow splitter 10 is adapted to the size of the expansion diameter tube 43. At the same time, since the protrusion ribs 133 protrude from the outer wall of the outer splitting member, several flow channels 47 are formed between the outer wall of the outer splitting member and the cavity wall of the accommodating cavity 430. In this embodiment, the inlet 11 of the airflow splitter 10 faces the first end 431, and the outlet 12 of the airflow splitter 10 faces the second end 432.

The homogeneous flow producing device 40 also includes a strainer assembly 45. the strainer assembly 45 is attached to the outlet 12 of the airflow splitter 10 and fixed in the second tube 44. The strainer assembly 45 includes a pressing block 451 and at least one strainer 452. In this embodiment, the pressing block 451 is in the shape of a hollow cylinder, and the pressing block 451 is interference-fitted in the second tube 44.

When the strainer assembly 45 includes a strainer 452, the strainer 452 is fixed on an end of the pressing block 451 facing the airflow splitter 10. And the strainer 452 abuts against edges of the outlet 12 of the airflow splitter 10.

The homogeneous flow producing device 40 also includes a flow sensor 46 accommodated in the second tube 44, and the flow sensor 46 is configured to measure a flow rate of the gas in the second tube 44. The airflow splitter 10 can make the gas flow homogeneously, which can improve the accurate measurement and the stability of homogeneous flow producing device 40.

The detail structure of the airflow splitter 10 can refer to the above-mentioned embodiments. Since the homogeneous flow producing device 40 adopts all the technical solutions of all the above-mentioned embodiments, it at least has all the beneficial effects brought by the technical solutions of the above-mentioned embodiments, which will not be repeated here.

Obviously, those skilled in the art can make various changes and modifications to the application without departing from the spirit and scope of the application. In this way, if these modifications and variations of this application fall within the scope of the claims of this application and their equivalent technologies, this application is also intended to include these modifications and variations.

The above-listed are only preferred embodiments of the application, and certainly cannot limit the scope of rights of the application. Therefore, equivalent changes made according to the claims of the application still fall within the scope of the application.

1. An airflow splitter, comprising:
an inlet;
an outlet; and
at least two splitting members, each splitting member comprising a small aperture end and a large aperture end opposite to each other, the small aperture ends of all the splitting members facing the inlet, and the large aperture ends of all the splitting members facing the outlet; a flow pathway defined between the small aperture ends and the large aperture ends, the at least two splitting members sleeved sequentially, in each two adjacent splitting members, the large aperture end of one splitting member being located in the small aperture end of the other splitting member, and an annular gap being formed between each two adjacent splitting members, and the annular gap being communicated with the flow pathway.

2. The airflow splitter according to claim 1, wherein the flow pathway is in a truncated cone shape, a central axis of the flow pathway of all the splitting members are coincident, and extension lines of outer walls of all the splitting members intersect at the same point.

3. The airflow splitter according to claim 1, wherein each two adjacent splitting members are constituted of an inner splitting member, and an outer splitting member located outside of the inner splitting member; an inclination of an outer surface of the inner splitting member is smaller than that of the outer splitting member; a diameter of the small aperture end of the inner splitting member is smaller than that of the outer splitting member; a diameter of the large aperture end of the inner splitting member is smaller than that of the outer splitting member.

4. The airflow splitter according to claim 3, wherein the small aperture end of the inner splitting member extends away from the outer splitting member and protrudes out of the outer splitting member; the large aperture end of the outer splitting member extends away from the inner splitting member and protrudes out of the inner splitting member.

5. The airflow splitter according to claim 4, wherein a plurality of protrusion ribs protrude from the outer wall of the outer splitting member at intervals and away from the inner splitting member, the protrusion ribs extends along a direction parallel to the central axis of the flow pathway.

6. The airflow splitter according to claim 5, wherein a plurality of limiting ribs protrude from the outer splitting member, the limiting ribs are arranged along a direction parallel to the central axis of the flow pathway, and the limiting ribs protrude from an inner wall of the outer splitting member towards the inner splitting member, and the limiting ribs abut against the outer wall of the outer splitting member.

7. The airflow splitter according to claim 5, wherein a plurality of auxiliary flow pathways are form between the inner splitting member and the outer splitting member.

8. The airflow splitter according to claim 2, wherein all the splitting members have same height along the central axis of the flow pathway.

9. An airflow splitting assembly, comprising:
an airflow splitter, the airflow splitter comprising:
an inlet;
an outlet;
a first splitting member; and
a second splitting member sleeved outside of the first splitting member; wherein each of the first splitting member and the splitting member having a small aperture end and a large aperture end opposite to each other, the small aperture ends of the first splitting member and the second splitting member facing the inlet, and the large aperture ends of the first splitting member and the second splitting member facing the outlet; a flow pathway defined between the small aperture ends and the large aperture ends, the large aperture end of the first splitting member located in the small aperture end of the second splitting member; an annular gap being formed between the first splitting member and the second splitting member, and the annular gap communicated with the flow pathway; and
a strainer, arranged at the outlet of the airflow splitter to enable the gas to pass through.

10. The airflow splitting assembly according to claim 9, wherein the flow pathway is in a truncated cone shape, a central axis of the flow pathway of the first and the second splitting members are coincident, and extension lines of outer walls of the first and the second splitting members intersect at the same point.

11. The airflow splitting assembly according to claim 9, wherein an inclination of an outer surface of the first splitting member is smaller than that of the second splitting member; a diameter of the small aperture end of the inner splitting member is smaller than that of the second splitting member; a diameter of the large aperture end of the first splitting member is smaller than that of the second splitting member.

12. The airflow splitting assembly according to claim 11, wherein the small aperture end of the first splitting member extends away from the second splitting member, and protrudes out of the second splitting member; the large aperture end of the second splitting member extends away from the first splitting member and protrudes out of the first splitting member.

13. The airflow splitting assembly according to claim 12, wherein a plurality of protrusion ribs protrude from the outer wall of the second splitting member and away from the first splitting member at the same interval, the protrusion ribs further extend along a direction parallel to the central axis of the flow pathway.

14. The airflow splitting assembly according to claim 13, wherein a plurality of limiting ribs protrude from an inner wall of the second splitting member and towards the first splitting member, the limiting ribs are arranged along a direction parallel to the central axis of the flow pathway, and the limiting ribs abut against the outer wall of the outer splitting member.

15. The airflow splitting assembly according to claim 11, wherein a plurality of auxiliary flow pathways are form between the inner splitting member and the outer splitting member.

16. The airflow splitting assembly according to claim 11, wherein all the splitting members have same height along the central axis of the flow pathway.

17. A homogeneous flow producing device, comprising:
a main body, defining a gas output mouth;
a first tube, attached to the main body and communicated with the gas output mouth;
a second tube, having a diameter lager than that of the first tube;
a expansion tube, having a first end, a second end opposite to each other, and defining an accommodating cavity between the first end and the second end, the first end is connected to the first tube, and the second end is connected to the second tube; and
an airflow splitter, received in the accommodating cavity, the airflow splitter comprising:
an inlet, facing to the first end;
an outlet, facing to the second end; and
at least two splitting member, each splitting member comprising a small aperture end and a large aperture end opposite to each other, the small aperture ends of all the splitting members facing the inlet, and the large aperture ends of all the splitting members facing the outlet; a flow pathway defined between the small aperture ends and the large aperture ends, the at least two splitting members sleeved sequentially, in two adjacent splitting members, the large aperture end of one splitting member located in the small aperture end of the other splitting member, and an annular gap being formed between each two adjacent splitting members, and the annular gap communicated with the flow pathway.

18. The homogeneous flow producing device according to claim 17, wherein the main body is a ventilator.

19. The homogeneous flow producing device according to claim 17, wherein the flow pathway is in a truncated cone shape, a central axis of the flow pathway of all the splitting members are coincident, and extension lines of outer walls of all the splitting members intersect at the same point.

20. The homogeneous flow producing device according to claim 17, wherein an auxiliary flow pathways is form between the inner splitting member and the outer splitting member.
